(19) United States
(12) Patent Application Publication
(10) Pub. No.: US 2010/0183542 A1
(43) Pub. Date: Jul. 22, 2010

(54) Synergism Between Activated Immune Cells and Conventional Cancer Therapies

(75) Inventors: Hung T. Khong, West Mobile, AL (US); Yixiang Wang, Mobile, AL (US); Soroosh Radfar, Mobile, AL (US)

Correspondence Address: THE WEBB LAW FIRM, P.C., 700 KOPPERS BUILDING, 436 SEVENTH AVENUE, PITTSBURGH, PA, 15219, US

(73) Assignee: UNIVERSITY OF SOUTH ALABAMA, Mobile, AL (US)

(21) Appl. No.: 12/447,027

(22) PCT Filed: Oct. 24, 2007
(86) PCT No.: PCT/US07/82377

§ 371 Date: Mar. 10, 2010

Related U.S. Application Data
(60) Provisional application No. 60/853,987, filed on Oct. 24, 2006.

(51) Int. Cl. A61K 38/20; A61K 38/21; A61K 38/19; A61K 35/26; A61P 35/00

(52) U.S. Cl. 424/85.2; 424/85.5; 424/85.1; 424/93.71

(57) ABSTRACT

The present invention relates to a composition, method and kit for treating cancer. The treatment comprises administering a pre-sensitizing agent to a tumor or subject, and administering a cancer therapy to the tumor cells or subject. The pre-sensitizing agent increases or induces the apoptosis of the tumor cells, as compared to the rate of apoptosis of tumor cells that have not been pre-sensitized prior to receiving the cancer therapy. In another aspect, the invention relates to a kit comprising a pre-sensitizing agent and a cancer therapy.

### BACKGROUND OF THE INVENTION

1. Field of the Invention

The present invention relates to compositions, methods and kits useful for treating cancer.

2. Background

A typical problem associated with currently available cancer treatments is the recurrence of the cancer, or metastasis of the cancer. Although the currently available treatments, such as surgery, chemotherapy and radiotherapy, are valid approaches to treat cancer, they do not successfully guard against the recurrence or metastasis of the cancer. Often, many patients die from recurrence or metastasis of cancer, or from the side effects associated with the radiotherapy or chemotherapy. For example, advanced breast cancer is often treated with chemotherapy such as doxorubicin, paclitaxel and docetaxel. However, patients often relapse within a few months, even with combination chemotherapy and/or radiotherapy treatments.

In the area of cancer treatment, there have been several unsuccessful attempts to enhance or improve the effects of chemotherapeutic agents by administering cytokines to a patient after the patient has been treated with chemotherapy. In all of these failed attempts, the combination of chemotherapy and cytokine treatment (also known as immunotherapy) has proven not to be any better than chemotherapy alone, or immunotherapy alone. Consequently, the United States Food and Drug Administration has refused to approve treatments combining chemotherapeutic agents with cytokines. Moreover, administering immunotherapy, particularly activated immune cells, before treating a subject with chemotherapy or radiotherapy was counter-intuitive in the art because it was thought that such an approach would destroy the administered immunotherapy.

### SUMMARY OF THE INVENTION

The present invention relates to discovery of a surprising synergism between activated immune cells, supernatant from activated immune cells or cytokines, and chemotherapy or radiotherapy. The tumor cells are pre-sensitized by a pre-sensitizing agent that is administered to a subject in need of a treatment for cancer or to the tumor cells. The pre-sensitizing agent is selected from the group consisting of activated immune cells, supernatant from activated immune cells, and cytokines. A chemotherapeutic drug is administered to the subject or the tumor cell either in sequence or simultaneously with the pre-sensitizing agent, thereby increasing or inducing apoptosis of the tumor cell. Alternatively or in addition to the administration of the chemotherapy, the tumor cells or subject can be treated with radiotherapy, thereby either increasing or inducing apoptosis of the tumor cell.

One aspect of the invention is a method of treating cancer comprising the steps of administering at least one cytokine to a subject, and administering a chemotherapeutic drug to the subject, thereby increasing or inducing apoptosis of at least one tumor cell. The cytokines may be selected from the group consisting of Il-2, Il-4, Il-6, Il-8, Il-10, Il-13, MN-7, TNF-α, Il-12p40, and GM-CSF. In another embodiment of this aspect, the cytokines may be selected from the group consisting of Il-6, Il-8, IFN-γ, TNF-α, and GM-CSF. In another embodiment of this aspect, the cytokines may be selected from the group consisting of IFN-γ, TNF-α, and GM-CSF. In another embodiment of this aspect, the cytokine comprises IFN-γ. The cytokine may further comprise TNF-α, Il-6, Il-8 or GM-CSF. The cytokine may further comprise TNF-α.

Another aspect of the invention is a method of treating cancer comprising the steps of administering at least one activated immune cell to a subject, and administering a chemotherapeutic drug to the subject, thereby increasing or inducing apoptosis of the tumor cell. The activated immune cell may be selected from the group consisting of CD4+ T cells, CD8+ T cells, natural killer cells, natural killer T cells, B cells, dendritic cells, peripheral blood mononuclear cells, neutrophils, monocytes, and macrophages. In one embodiment of this aspect, the activated immune cell is an activated CD4+ T cells. In another embodiment of this aspect, the activated immune cell is an activated peripheral blood mononuclear cell.

Another aspect of the invention is the method of treating cancer comprising the steps of administering a pre-sensitizing agent to a subject, allowing the pre-sensitizing agent to pre-sensitize at least one tumor cell, and treating the tumor cell or subject with radiotherapy. In one embodiment of this aspect, the radiotherapy may be selected from the group consisting of gamma irradiation and radiofrequency ablation. In another embodiment of this aspect, the pre-sensitizing agent in this aspect may be selected from the group consisting of activated immune cells, supernatant from the activated immune cells, and cytokines. The activated immune cell may be selected from the group consisting of CD4+ T cells, CD8+ T cells, natural killer cells, natural killer T cells, B cells, dendritic cells, peripheral blood mononuclear cells, neutrophils, monocytes, and macrophages. In one embodiment of this aspect, the activated immune cell is an activated CD4+ T cells. In another embodiment of this aspect, the activated immune cell is an activated peripheral blood mononuclear cell. The cytokines may be selected from the group consisting of Il-2, Il-4, Il-6, Il-8, Il-10, Il-13, IFN-γ, TNF-α, Il-12p40, and GM-CSF. In another embodiment of this aspect, the cytokines may be selected from the group consisting of Il-6, Il-8, IFN-γ, TNF-α, and GM-CSF. In another embodiment of this aspect, the cytokines may be selected from the group consisting of IFN-γ, TNF-α, and GM-CSF. In another embodiment of this aspect, the cytokine comprises IFN-γ. The cytokine may further comprise TNF-α, Il-6, Il-8 or GM-CSF. The cytokine may further comprise TNF-α.

Another aspect of the invention is a method of enhancing the activity of a chemotherapeutic drug administered in combination with a pre-sensitizing agent and administering said chemotherapeutic drug to the subject containing said tumor cell, wherein said administering is effective to produce at least about a two-fold and up to about a ten-fold reduction in vivo tumor volume relative to that provided by administering the chemotherapeutic drug or pre-sensitizing agent alone. The pre-sensitizing agent in this aspect may be selected from the group consisting of activated immune cells, supernatant from the activated immune cells, and cytokines. The activated immune cell may be selected from the group consisting of CD4+ T cells, CD8+ T cells, natural killer cells, natural killer T cells, B cells, dendritic cells, peripheral blood mononuclear cells, neutrophils, monocytes, and macrophages. In one embodiment of this aspect, the activated immune cell is an activated CD4+ T cell. In another embodiment of this aspect, the activated immune cell is an activated peripheral blood mononuclear cell. The cytokines may be selected from the group consisting of Il-2, Il-4, Il-6, Il-8, Il-10, Il-13, IFN-γ, TNF-α, Il-12p40, and GM-CSF. In another embodiment of this aspect, the cytokines may be selected from the group consisting of Il-6, Il-8, IFN-γ, TNF-α, and GM-CSF. In another embodiment of this aspect, the cytokines may be selected from the group consisting of IFN-γ, TNF-α, and GM-CSF. In another embodiment of this aspect, the cytokine comprises IFN-γ. The cytokine may further comprise TNF-α, Il-6, Il-8 or GM-CSF. The cytokine may further comprise TNF-α.

Another aspect of the invention is a kit for enhancing the activity of a cancer treatment comprising a pre-sensitizing agent and a chemotherapeutic agent. One embodiment is a kit wherein the pre-sensitizing agent is administered to a subject or tumor cell prior to the administration of the chemotherapeutic agent.

Another aspect of the invention is a method of treating cancer comprising administering a pre-sensitizing agent to a subject or tumor cell and administering a chemotherapeutic agent selected from the group consisting of dacarbazine, temozolomide, carboplatin, paclitaxel, cisplatin, vinblastine, and fluorouracil.

Another aspect of the invention is a method of treating cancer comprising administering a pre-sensitizing agent to a subject or tumor cell and administering a chemotherapeutic agent, wherein the cancer is selected from the group consisting of melanoma, breast cancer, prostate cancer, glioma, ovarian, cervical, lung, head and neck, and colon cancer.

Other applications and advantages afforded by the present invention will be apparent from the detailed description and exemplification hereinbelow.

### BRIEF DESCRIPTION OF THE DRAWING(S)

FIG. 1 depicts the results from experiments to determine the optimal amount of TMZ to treated A375 melanoma cells.

FIG. 2 depicts the results from the experiments to determine the optical amount of carboplatin to treat A375 melanoma cells.

FIG. 3 depicts the percentage of live cells after being treated with various concentrations of CD4+ T cells, and pre-sensitized at various concentration of CD4+ T cells and thereafter treated with TMZ.

FIG. 4 depicts the percentage of live cells after being treated with various concentrations of CD4+ T cells, and pre-sensitized at various concentration of CD4+ T cells and thereafter treated with carboplatin.

FIG. 5 depicts the percentage of live A375 melanoma cells and pre-sensitized live cells as a function of the dose of TMZ after 24 hours of being treated with the chemotherapeutic agent.

FIG. 6 depicts the percentage of live A375 melanoma cells and pre-sensitized live cells as a function of the dose of carboplatin after 24 hours of being treated with the chemotherapeutic agent.

FIG. 7 depicts the percentage of live 526 melanoma cells after being treated with a chemotherapeutic agent, activated CD4+ T cells, and after the cells were pre-sensitized with activated CD4+ T cells and thereafter treated with the chemotherapeutic agent.

FIG. 8 depicts the percentage of live 1938 melanoma cells after being treated with a chemotherapeutic agent, activated CD4+ T cells, and after the cells were pre-sensitized with activated CD4+ T cells and thereafter treated with the chemotherapeutic agent.

FIG. 9 depicts the percentage of live MCF-7 breast cancer cells after being treated with a chemotherapeutic agent, activated CD4+ T cells, and after the cells were pre-sensitized with activated CD4+ T cells and thereafter treated with the chemotherapeutic agent.

FIG. 10 depicts the percentage of live MDA MB-231 breast cancer cells after being treated with a chemotherapeutic agent, activated CD4+ T cells, and after the cells were pre-sensitized with activated CD4+ T cells and thereafter treated with the chemotherapeutic agent.

FIG. 11 depicts the percentage of live HCT116 colon cancer cells after being treated with a chemotherapeutic agent, activated CD4+ T cells, and after the cells were pre-sensitized with activated CD4+ T cells and thereafter treated with the chemotherapeutic agent.

FIG. 12 depicts the percentage of live DU145 prostate cancer cells after being treated with a chemotherapeutic agent, activated CD4′ T cells, and after the cells were pre-sensitized with activated CD4+ T cells and thereafter treated with the chemotherapeutic agent.

FIG. 13 depicts the results from an apoptosis assay.

FIG. 14 depicts the results from a transwell experiment.

FIG. 15 and FIG. 16 depict the amount of cytokines observed in the supernatant of activated CD4+ T cells cultured for 48 hours alone (row 1), or cultured for 24 hours alone and an additional 24 hours in the presence of temozolomide (row 2).

FIG. 17 depicts the results from an experiment testing the effect on apoptosis of A375 melanoma cells that were pre-sensitized with the combination of Il-2 and Il-4 (“G1”).

FIG. 18 depicts the results from an experiment testing the effect on apoptosis of A375 melanoma cells that were pre-sensitized with the combination of Il-6 and Il-8 (“G2”).

FIG. 19 depicts the results from an experiment testing the effect on apoptosis of A375 melanoma cells that were pre-sensitized with the combination of Il-10, Il-12p40 and Il-13 (“G3”).

FIG. 20 depicts the results from an experiment testing the effect on apoptosis of A375 melanoma cells that were pre-sensitized with the combination of IFN-γ, TNF-α and GM-CSF (“G4”).

FIG. 21 depicts the results from an experiment testing the effect on apoptosis of A375 melanoma cells that were pre-sensitized with the combination of all cytokines.

FIG. 22 depicts the results from a transwell experiment testing the effect on apoptosis of A375 melanoma cell pre-sensitized with supernatant from CD4+ T cells.

FIG. 23 is an image of an electrophoretic gel detecting the presence of cytokine receptors in various cell lines.

FIG. 24 shows the cell inhibition in Hela cells treated by activated CD4+ T cells at various doses of radiation.

FIG. 25 shows the cell inhibition of Hela cells treated by activated CD4+ T cells directly, via transwell or supernatant derived from activated CD4+ T cells, followed by radiation.

FIG. 26 shows the effect of pre-sensitization of Hela cells by supernatant from activated CD4+ T cells when the cells are treated with gamma irradiation.

FIG. 27 a-i depict the cell inhibition of various cancer cell lines when treated with gamma irradiation, pre-sensitized with activated CD4+ T cell or their supernatants, followed by various doses of radiation.

FIG. 28 depicts the results from flow cytometric analysis of apoptosis of LN229 pre-sensitized by supernatant from activated CD4+ T cells and treated by gamma irradiation.

FIG. 29 depicts the effect of individual and combination of cytokines on cell inhibition after treatment with gamma irradiation.

FIG. 30 depicts results from experiments to test the ability of IFN-γ to enhance cytotoxicity in gamma irradiated Hela cells.

FIG. 31 depicts the cell inhibition of Hela cells treated with IFN-γ and IFN-γ combined with other cytokines.

FIG. 32 depicts the cell inhibition of Hela cells treated with IFN-γ and IFN-γ combined with other cytokines.

FIG. 33 depicts the cell inhibition of Hela cells treated IFN-γ or IFN-γ in combination with other cytokines followed by gamma irradiation.

FIG. 34 depicts the synergistic effect of combining IFN-γ, TNF-α, Il-2 and Il-6 to pre-sensitize Hela cells before gamma irradiation.

FIG. 35 depicts the cell inhibition of Hela cells pre-sensitized by various cytokines or combinations of cytokines followed by gamma irradiation.

FIG. 36 depicts results of cell inhibition in other cancer cell lines similar to the results observed in the Hela cells.

FIG. 37 depicts the results from experiments testing the effectiveness of chemotherapeutic drugs on A375 melanoma cells that were pre-sensitized with activated CD4+ T cells and CD4− PBMC.

FIG. 38 is a graph depicting the A375 melanoma tumor volume from an in vivo experiment showing the increased effect of chemotherapy (temozolomide) in pre-sensitized tumor cells with activated CD4+ T cells.

FIG. 39 is a graph depicting the MDA-MB 231 breast cancer tumor volume from an in vivo experiment showing the increased effect of chemotherapy (taxol) in pre-sensitized tumor cells with activated CD4+ T cells.

### DETAILED DESCRIPTION OF THE INVENTION

The terms “chemotherapy,” “chemotherapeutic agent”, “chemotherapeutic drug”, or any derivation thereof refers to any form of treating cancer with a drug or composition that kills a cancer cell. Non-limiting examples of “chemotherapeutic agents” include conventional chemotherapeutic drugs such as alkylating agents (for example cisplatin and carboplatin), plant alkaloids and temenoids (for example taxanes, vinca alkaloids (for instance, vinblastine, vincristine, vinorelbine and vindesine), and podophyllotoxin), anti-metabolites (for example, azathioprine and mercaptopurine); topoisomerase inhibitors; monoclonal antibodies; hormones; anti-tumor antibiotics; chemoembolization; targeted therapy (such as bevacizumab) and cryosurgery.

The terms “radiotherapy” or any derivation thereof refers to any form of cancer treatment that uses electromagnetic waves or heat energy in any form that is used to kill cancer cells. Examples of radiotherapy include, but are not limited to, gamma irradiation and radiofrequency ablation.

The present invention provides a method of treating cancers by pre-sensitizing tumor cells, then administering known cancer therapies such as chemotherapy or radiotherapy. Immunotherapy is used to sensitize tumor cells to the cytotoxic effect of the chemotherapy or radiotherapy. This strategy employs the use of activated immune cells to sensitize tumor cells prior to or simultaneously to the administration of the chemotherapeutic agents that are clinically active against that particular tumor will be administered, or prior to the administration of radiotherapy. In addition, activated immune cells may exert direct activity on tumor cells through apoptotic pathways such as the engagement of Fas ligand, which is highly expressed on activated T cells, and Fas receptor on tumor cells. This model does not depend on the antigen-specific activation of immune cells, and therefore will be applicable to all tumor types and all patients regardless of their HLA status. In addition, it is much easier and much less expensive to use in the clinic. However, specific immune cells may be used.

This chemoimmunotherapy or radioimmunotherapy strategy may not only exert dramatic anti-tumor responses but also may prevent recurrence because of the potential generation of memory tumor-antigen specific T cells. Multiple cytokines and other soluble factors released from the immune cells in the tumor microenvironment, possibly other interactions between the immune cells and the tumor cells, and tumor antigens released from chemotherapy- or radiotherapy-induced cell death may activate and expand tumor infiltrating immune cells, thereby generating and/or potentiating a successful adaptive immune response against cancer. This may be considered an effective form of in situ vaccination.

Many cancers may be treated by the disclosed invention. Some non-limiting examples of cancers that can be treated by this invention include melanoma, breast cancer, prostate cancer, glioma, ovarian, cervical, lung, head and neck, and colon cancer.

The present invention involves administering a pre-sensitizing agent that pre-sensitizes tumors cells to cancer treatments, and administering a cancer treatment to increase or induce apoptosis of the tumor cells.

### I. PRE-SENSITIZING AT LEAST ONE TUMOR CELL

The tumor cells can be pre-sensitized by administering immune cells, supernatant derived from immune cells, or by administering cytokines.

The administration of any immune cell may pre-sensitize the tumor cells to a cancer treatment. For example, when tumor cells are pre-sensitized with activated CD4+ T cells or non-specific activated CD4+ T cells, the tumor cells were much more susceptible to cancer treatments. Other examples of immune cells that may pre-sensitize tumor cells include CD8+ T cells, natural killer cells, natural killer T cells, B cells, dendritic cells, peripheral blood mononuclear cells, neutrophils, monocytes or macrophages. The immune cells used to pre-sensitize tumor cells must be activated, and can be either non-specific or specific.

Another aspect of the invention is to administer supernatant derived from an immune cell to pre-sensitize a tumor cell. The supernatant can be derived by any method known within the art.

In another aspect of the invention, the pre-sensitizing agent is at least one cytokine. In another aspect of the invention, the cytokine used to pre-sensitize the tumor cells may be selected from the group consisting of Il-2, Il-4, Il-6, Il-8, Il-10, Il-13, IFN-γ, IFN-α, Il-12p40, and GM-CSF. The cytokine may alternatively be a combination of IFN-γ and a cytokine selected from the group consisting of TNF-α, Il-2, Il-4, Il-6, Il-8, Il-10, Il-12p40, Il-13, and GM-CSF; or a combination of IFN-γ, TNF-α and a cytokine selected from the group consisting of Il-2, Il-4, Il-6, Il-8, Il-10, Il-13, Il-12p40, and GM-CSF. The cytokine may alternatively be selected from the group consisting of Il-6, Il-8, IFN-γ, TNF-α and GM-CSF. The cytokine may alternatively be selected from the group consisting of IFN-γ, IFN-α and GM-CSF. Another embodiment of this aspect is pre-sensitizing the tumor cells with a pre-sensitizing agent comprising IFN-γ. In this aspect, the cytokine may further comprise TNF-α, Il-6, Il-8 or GM-CSF.

The methods of administering the pre-sensitizing agents are known within the art. Some examples of routes of administration include intra-tumoral, oral, subcutaneous, intraperitoneal, and intravenous. The pre-sensitizing agents may also be administered into the loci where tumor cells have been removed or substitute the volume of the tumor cells that have been removed.

The pre-sensitizing agent can be administered together or in sequence with the cancer therapy. If the pre-sensitizing agent is administered in sequence with the cancer therapy, it can be administered anytime from immediately after the administration of the pre-sensitizing agent to 10 days after the administration of the pre-sensitizing agent; from 24 hours after the administration of the pre-sensitizing agent to 5 days after the administration of the pre-sensitizing agent; or from 48 hours after the administration of the pre-sensitizing agent to 5 days after the administration of the pre-sensitizing agent.

### II. ADMINISTERING A CANCER THERAPY

The subject or tumor cells are additionally treated with a cancer therapy applicable to the particular form of cancer inflicting the subject. In one aspect of the invention, the cancer therapy is chemotherapy. In this aspect, the chemotherapeutic agent is administered to the subject or tumor cells in a manner known to a skilled artisan. For example, in applicable situations, the chemotherapeutic agent is administered intra-tumorally. The chemotherapeutic agent selected should be appropriate to treat the particular form of cancer or tumor cells inflicting the subject. In one embodiment of this aspect, the chemotherapeutic agent is selected from the group consisting of dacarbazine, temozolomide, carboplatin, paclitaxel, cisplatin, vinblastine and fluorouracil.

The chemotherapy can be administered in sequence or simultaneously with the administration of the pre-sensitizing agent. If the chemotherapy is administered in sequence to the administration of the pre-sensitizing agent, it should be administered within 10 days, within 7 days, or within 5 days following the administration of the pre-sensitizing agent. The chemotherapy may be administered in sequence, or immediately after the administration of the pre-sensitizing agent, the day after the administration of the pre-sensitizing agent, 24 hours after the administration of the pre-sensitizing agent, 48 hours after the administration of the pre-sensitizing agent, or 72 hours following the administration of the pre-sensitizing agent.

In another aspect of the invention, the tumor cells or subject are subjected to a chemotherapeutic agent and radiotherapy within 7 days following the administration of the pre-sensitizing agent. In one embodiment of this aspect, the pre-sensitizing agent and the chemotherapeutic agent are administered simultaneously. Tumor cells are allowed to pre-sensitize over the course of at least 24 hours, but not more than 7 days following the administration of the pre-sensitizing agent. After the tumor cells have been pre-sensitized, radiotherapy is administered to the subject or tumor cells. The radiotherapy is administered no earlier than 24 hours following the administration of the pre-sensitizing agent, and no later than 7 days following the administration of the pre-sensitizing agent.

Another embodiment of this aspect is administering a pre-sensitizing agent to at least one tumor cell or a subject, and then treating the subject or tumor cell with chemotherapy and radiotherapy, wherein the chemotherapeutic agent is administered in sequence to the pre-sensitizing agent. The chemotherapeutic agent can be administered together with the pre-sensitizing agent, after the administration of the pre-sensitizing agent, the day following the administration of the pre-sensitizing agent, 24 hours following the administration of the pre-sensitizing agent, 48 hours following the administration of the pre-sensitizing agent, or 72 hours following the administration of the pre-sensitizing agent. The chemotherapeutic agent should be administered during the time that the tumor cells are pre-sensitized. Thus, the chemotherapeutic agent should be administered within 10 days, within 7 days, or within 5 days following the administration of the pre-sensitizing agent. The radiotherapy can be administered as early as immediately after the administration of the pre-sensitizing agent, the day following the administration of the pre-sensitizing agent, 24 hours following the administration of the pre-sensitizing agent, 48 hours following the administration of the pre-sensitizing agent, or 72 hours following the administration of the pre-sensitizing agent. The radiotherapy should be administered during the time that the tumor cells are pre-sensitized. Thus, the radiotherapy should be administered within 10 days, within 7 days or within 5 days following the administration of the pre-sensitizing agent.

### III. KITS FOR TREATING CANCER

Another aspect of the invention is a kit for enhancing the activity of a cancer treatment. The kit comprises a pre-sensitizing agent and a cancer treatment. The kit may require that the pre-sensitizing agent be administered before the cancer treatment.

In one embodiment of this aspect, the pre-sensitizing agent is at least one activated immune cell, a supernatant from at least one activated immune cell, or at least one cytokine. The activated immune cell can be specific or non-specific, or can be selected from the group consisting of CD4+ T cells, CD8+ T cells, natural killer cells, natural killer T cells, 13 cells, dendritic cells, peripheral blood mononuclear cells, neutrophils, monocytes and macrophages.

The cytokines may be selected from the group consisting of Il-2, Il-4, Il-6, Il-8, Il-10, Il-13, IFN-γ, TNF-α, Il-12p40, and GM-CSF. The cytokine may alternatively be a combination of IFN-γ and a cytokine selected from the group consisting of TNF-α, Il-2, Il-4, Il-6, Il-8, Il-10, Il-12p40, Il-13, and GM-CSF; or a combination of IFN-γ, TNF-α and a cytokine selected from the group consisting of Il-2, Il-4, Il-6, Il-8, Il-10, Il-13, Il-12p40, and GM-CSF. The cytokine may alternatively be selected from the group consisting of Il-6, Il-8, IFN-γ; TNF-α and GM-CSF. The cytokine may alternatively be selected from the group consisting of IFN-γ, TNF-α and GM-CSF. Another aspect of the invention is pre-sensitizing the tumor cells with a pre-sensitizing agent comprising IFN-γ. In this aspect, the cytokine may further comprise TNF-α, Il-6, Il-8 or GM-CSF.

The cancer treatment may be a chemotherapeutic agent. Chemotherapeutic agent may be selected from a group consisting of dacarbazine, temozolomide, carboplatin, paclitaxel, cisplatin, vinblastine, and fluorouracil.

### IV. EXPERIMENTS

The experiments discussed below relate to CD4+ T cell and CD4− peripheral blood mononuclear cells. Other activated immune cells should have similar effects. Some of these other immune cells are CD8+ T cells, natural killer cells, natural killer T cells, B cells, dendritic cells, peripheral blood mononuclear cells, neutrophils, monocytes and macrophages.

### Experiment 1

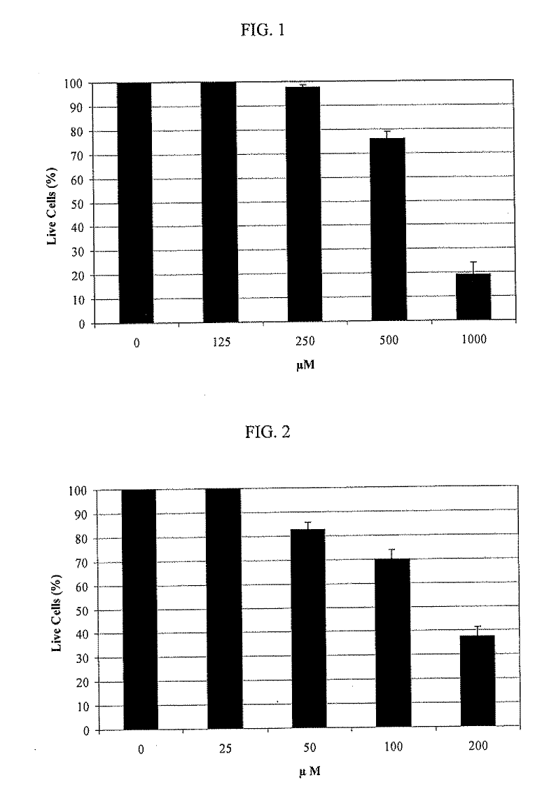

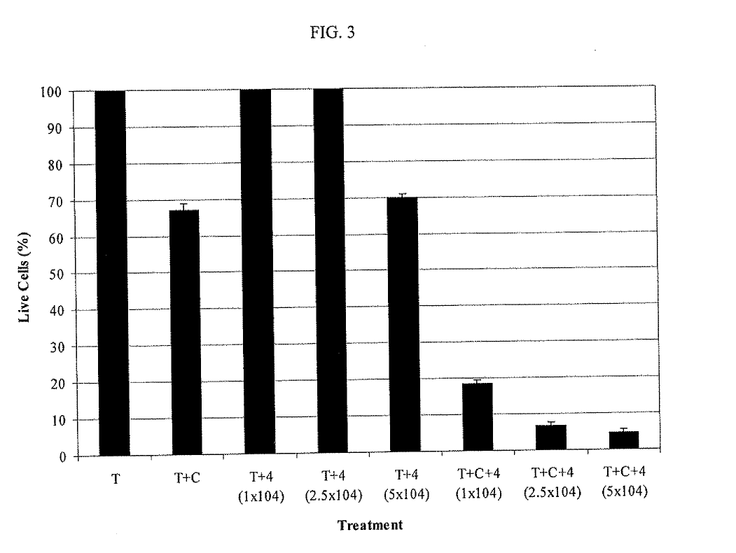

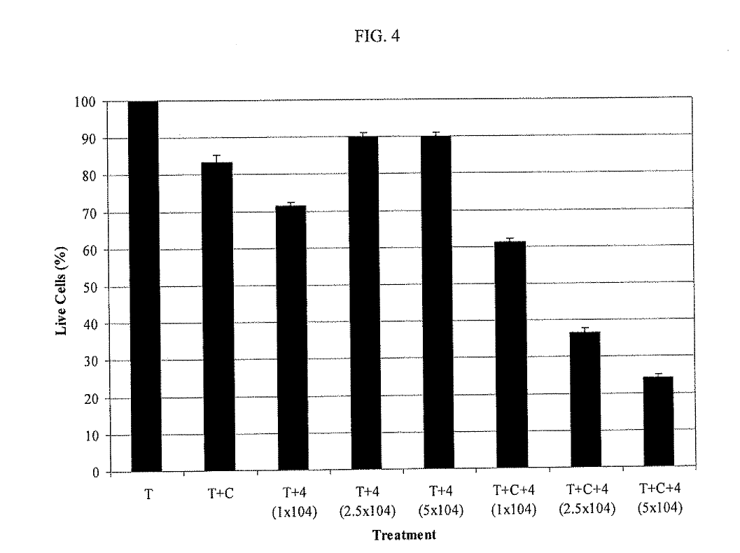

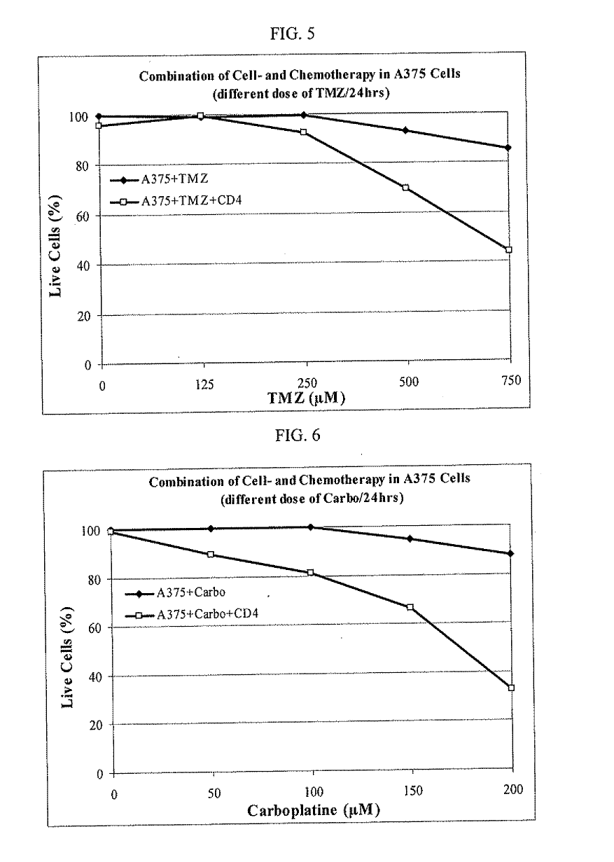

Various tumors cells were tested to determine if the pre-sensitization of the tumor cells with non-specific activated CD4+ T cells would increase or induce apoptosis of the tumor cells. First, optimal doses of TMZ (see FIG. 1) and carboplatin (see FIG. 2) were determined. Next, optimal concentrations of the CD4+ T cells were determined (see FIG. 3, FIG. 4, FIG. 5 and FIG. 6). After determining the optimal doses and concentrations, various tumor cells were pre-sensitized with activated CD4+ T cells and then treated with either TMZ or carboplatin in controlled experiments. The tumor cells used were A375 melanoma cells, 526 melanoma cells, 1938 melanoma cells, MCF-7 breast cancer cells, MDA MB-231 breast cancer cells, colon cancer cells, and prostate cancer cells.

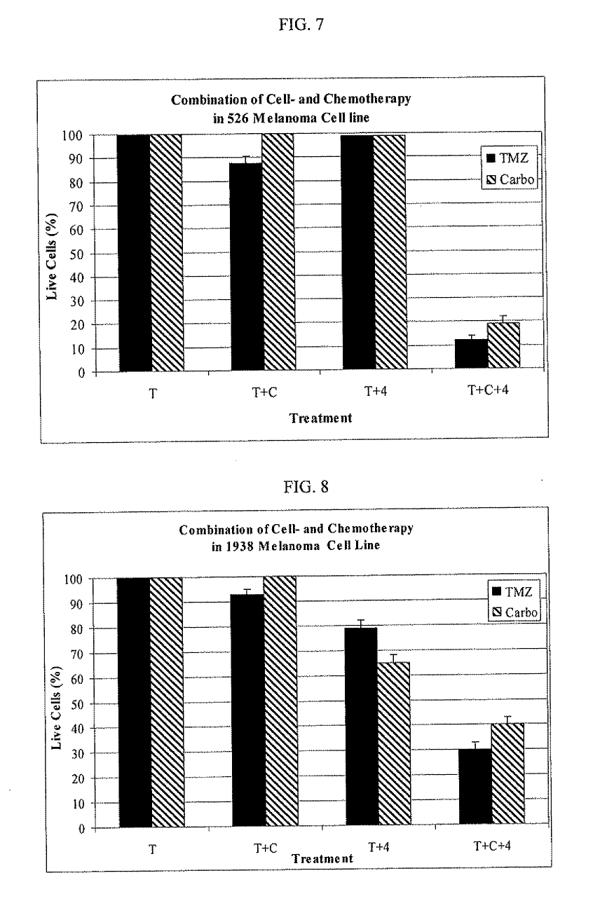

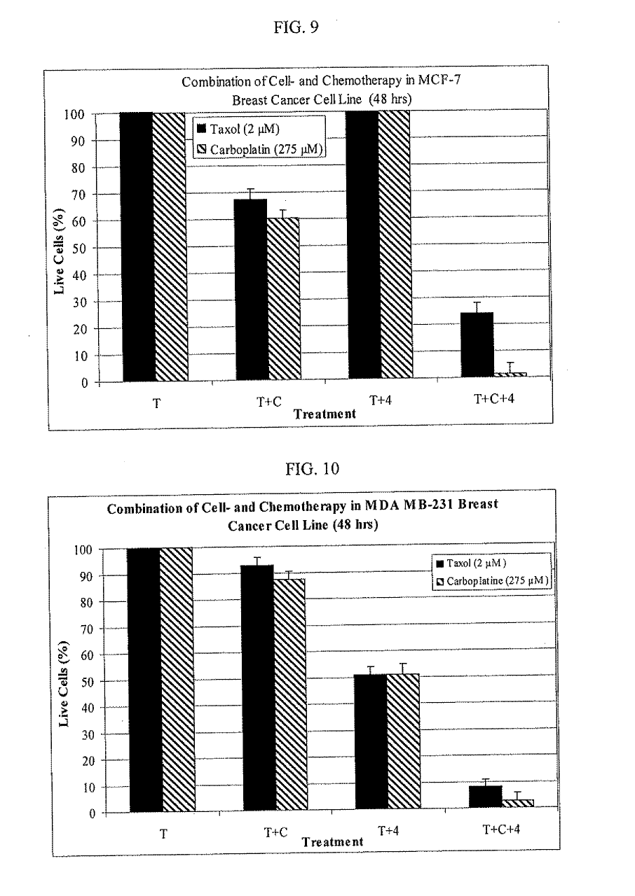

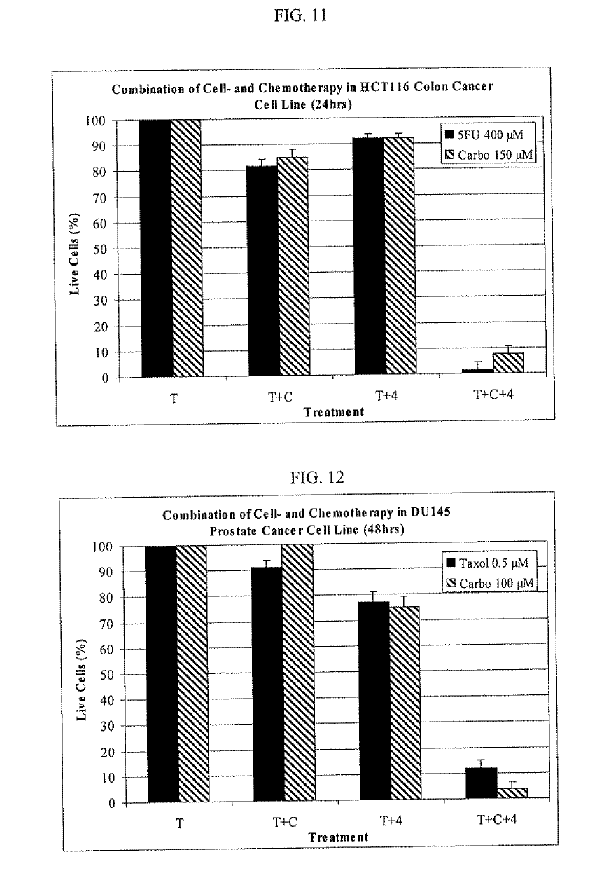

The tumor cells were cultured overnight at 1×10⁴ cells per well in a 96-well flat bottom plate in triplicate. CD4+ T cells were activated by coating non-tissue culture in a 24-well plate with 1 ml of OKT3 (5 μg/ml) and CD28 (1 μg/ml) and incubated overnight at 4° C. in 1 ml PBS. The plated tumor cells were identified as either tumor cells alone, tumor cells treated with the chemotherapeutic agent (TMZ at 750 μM or carboplatin at 150 μM), tumor cells treated with activated CD4+ T cells, or tumor cells pre-sensitized with activated CD4+ T cells and treated with the chemotherapeutic agent (TMZ at 750 μM or carboplatin at 150 μM). For each well designated as receiving activated CD4+ T cells, 2.5×10⁴ activated CD4+ T cells were added to those wells. The cells were incubated for 24 hours. The chemotherapeutic drug was added to wells designated as receiving a chemotherapeutic drug, and the plates were incubated for 24 or 48 hours, depending on the tumor cell line used. After incubation was completed, 100 μL of the medium was removed and 10 μL of WST-1 cell proliferation reagent (Roche Diagnostics) was added. The plates were incubated for 0.5 to 4 hours. Following incubation, the plates were processed by a plate reader at 450 nm. The percentages of live cells were calculated from the results of the plate reader and are shown in FIG. 7 through FIG. 12.

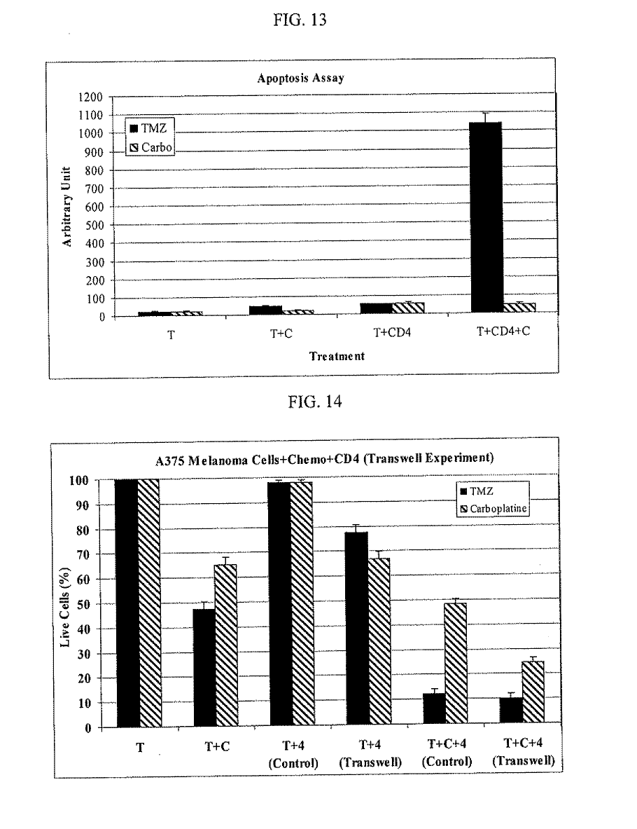

In sum, the results from the experiments evidence that pre-sensitization of tumor cells from various histologies with activated CD4+ T cells increases or induces apoptosis of the tumor cells. An apoptosis assay confirmed that the apoptotic pathway is the pathway that the pre-sensitizer tumors die (see FIG. 13).

### Experiment 2

A375 melanoma cells were plated at 4×10⁴ per well in 0.8 ml of complete medium in a 24-well plate overnight. The following day, the cells were treated with 2.5×10⁴ activated CD4+ T cells either directly or via transwell. The CD4+ T cells were activated according to the procedure described in Experiment 1. The plate was incubated for 2 days. The pre-sensitized cells were treated with either TMZ at 750 μM or carboplatin at 150 μM.

The results, shown in FIG. 14, evidence that soluble factors secreted by the activated CD4+ T cells play a role in pre-sensitizing the tumor cells to the chemotherapeutic agents.

### Experiment 3

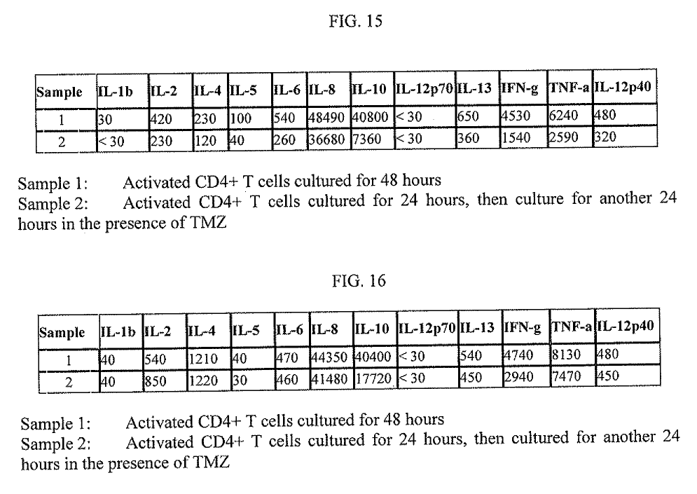

Various combinations of cytokines were tested as possible pre-sensitizing agents for tumor cells. Optimal doses of cytokines were determined by studying the cytokines secreted from activated CD4+ T cells (see FIG. 15 and FIG. 16). A375 melanoma cells were cultured overnight at 1×10⁴ cells per well in a 96-well flat bottom plate in triplicate. The plated tumor cells were identified as either tumor cells alone, tumor cells treated with the chemotherapeutic agent (TMZ at 750 μM or carboplatin at 150 μM), tumor cells treated with cytokines, or tumor cells pre-sensitized with cytokines and treated with the chemotherapeutic agent. The tumor cells that were treated with cytokines were either treated with an optimal dose of cytokines or a double dose of cytokines, as determined by the results shown in FIG. 13 and FIG. 16, which are results from experiments studying the concentrations of various cytokines secreted by activated CD4+ T cells. The experiment was repeated several times with different combinations of cytokines. The combinations of cytokines were: Il-2 and Il-4 (“G1”), Il-6 and Il-8 (“G2”), Il-10, Il-12p40 and Il-13 (“G3”), and IFN-γ, TNF-α and GM-CSF (“G4”).

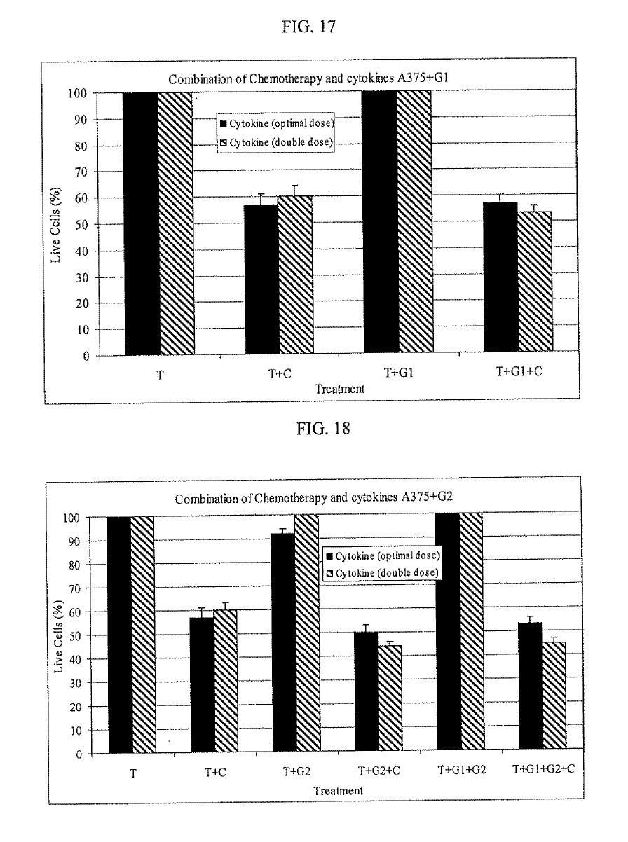

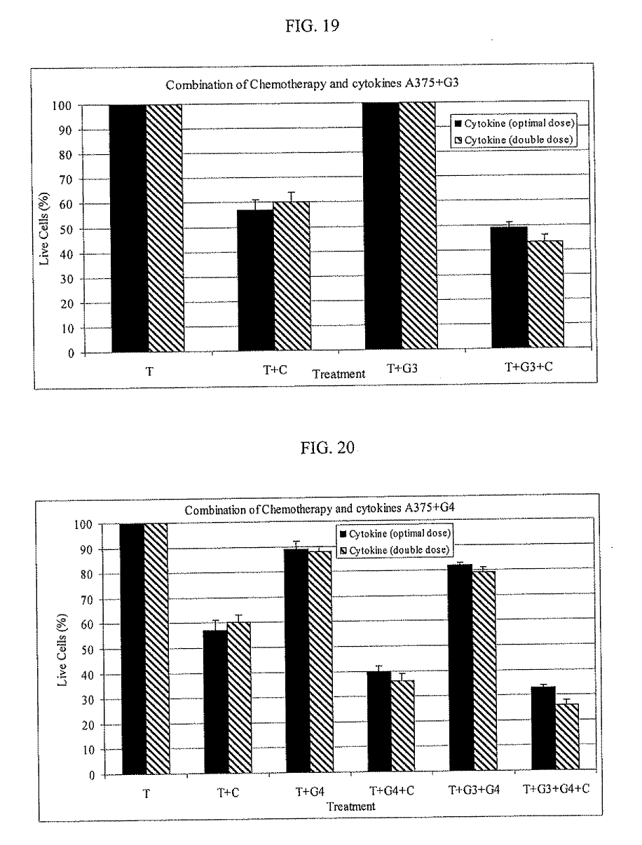

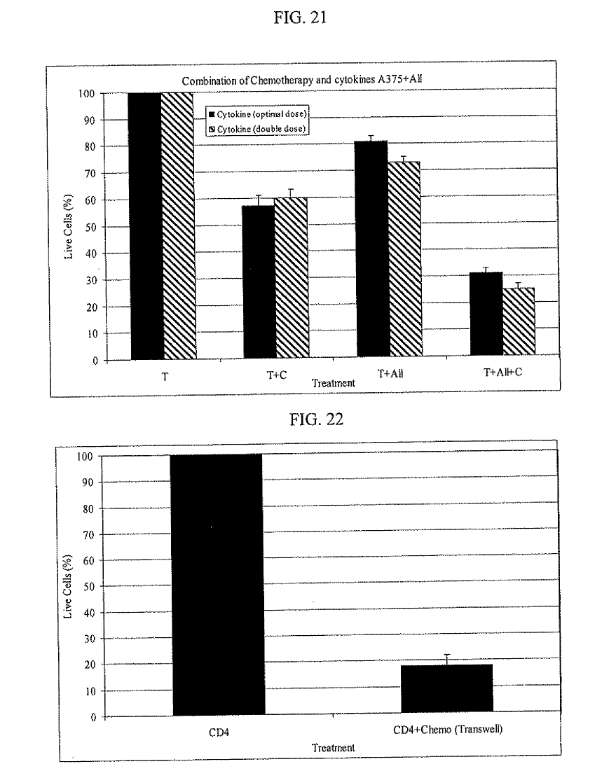

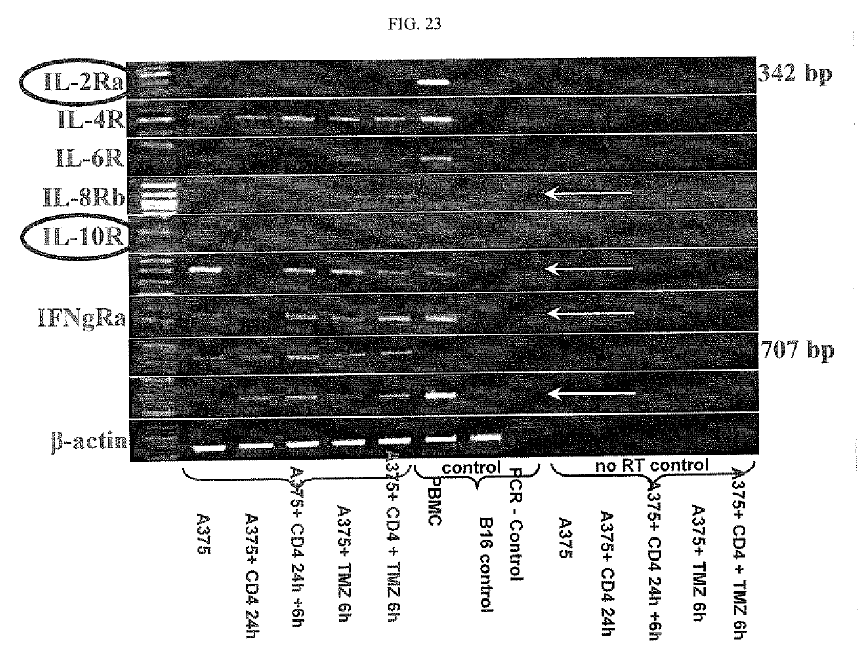

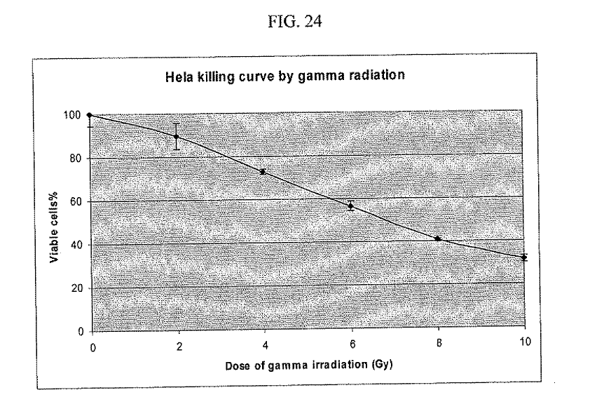

The results, which are shown in FIG. 17 through FIG. 22, evidence that cytokines alone increase or induce apoptosis of tumor cells.

### Experiment 4

Hela cells were plated at 4×10⁴ cells per well in 0.8 ml of complete medium in 24-well plate overnight. Activated CD4+ T cells were prepared according to the procedure described in Experiment 1. The following day Hela cells were treated with 1×10⁴ cells or 2×10⁴ cells of activated CD4+ T cells directly (designated as 1e4 (1×10⁴ T cells) or 2e4 (2×10⁴ T cells)), via transwell (designated as 1e4tr (1×10⁴ T cells) or 2e4 (2×10⁴ T cells)), or via supernatant derived from 1×10⁴ T cells (1e4s) or 2×10⁴ T cells (2e4s) for 2 days. The transwell CD4+ T cells had no direct contact with the Hela cells, but the soluble factors secreted by the CD4+ T cells could pass through the transwell membrane and interact with the Hela cells. The pretreated Hela were irradiated with 4Gy and plated at 5×10³ cells per well with 200 μl of fresh complete medium in triplicate. On day 5, viability was measured by WST-1 reagent.

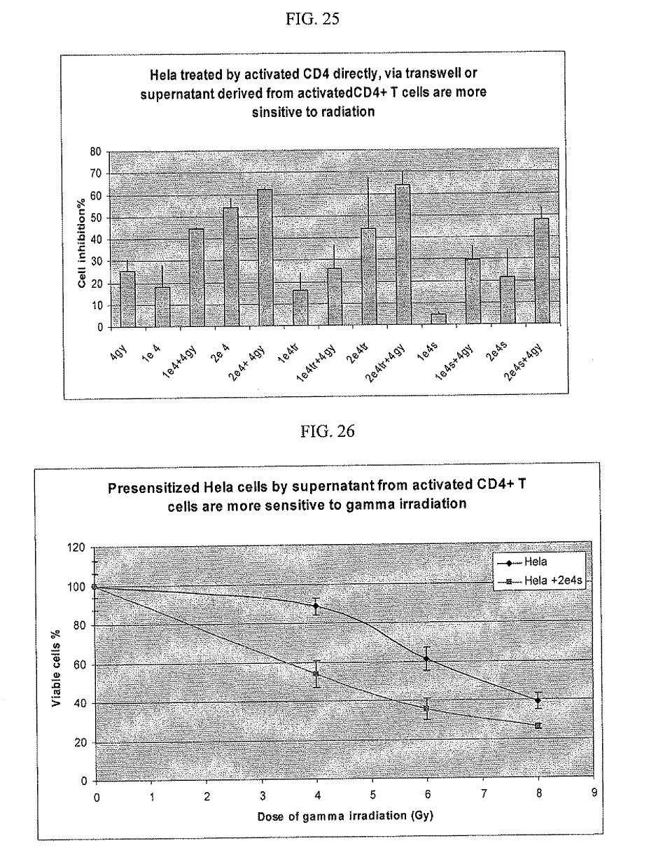

The results show that soluble factors are important for cell inhibition and radio-sensitivity, but not cell-cell contact (see FIG. 25 and FIG. 26). Results are means±SEM. Similar results were seen in another experiment.

### Experiment 5

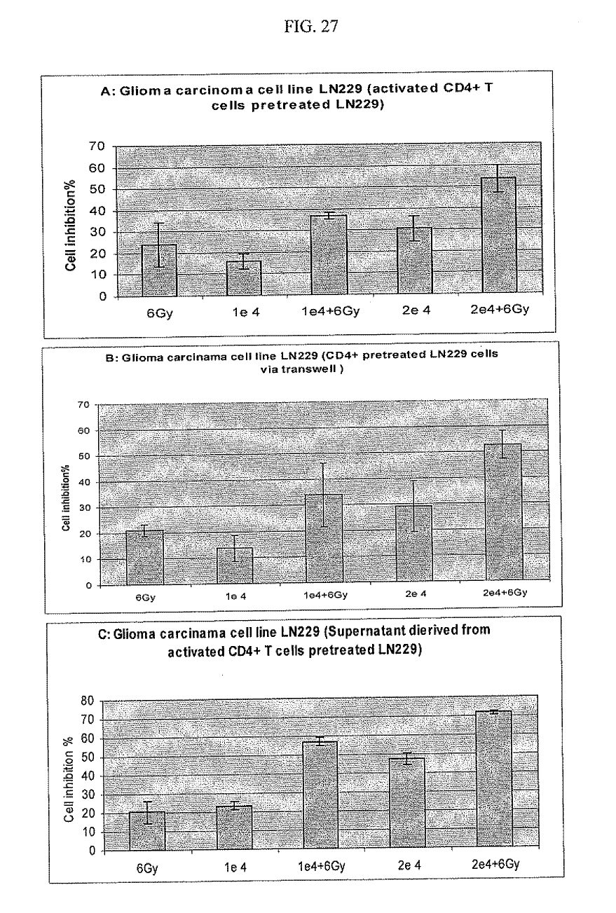

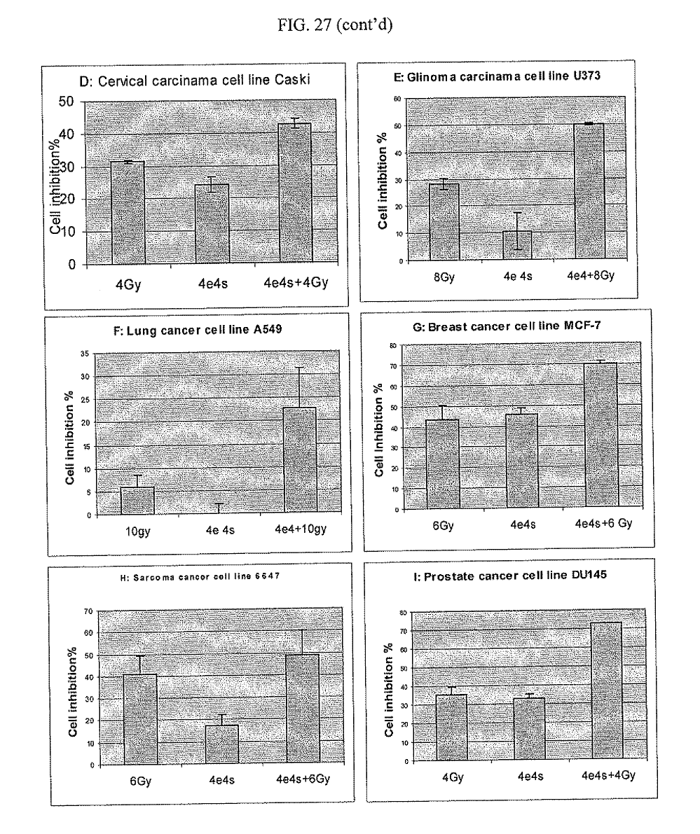

To verify the principle that pre-sensitized tumor cells are more susceptible to radiotherapy, other cancer cell lines were tested. Human glioma cell line LN229 was treated by a different dose of activated CD4+ T cells (see FIG. 27 A), CD4+ T cells soluble factors permeable through the pores in the transwell (see FIG. 27 B), or supernatant from activated CD4+ T cells (see FIG. 27 C), and then irradiated with 6Gy of gamma radiation similar to the procedure in Experiment 4.

The results showed a similar pattern as the results of Experiment 4. Pre-sensitized LN229 cells were more susceptible to gamma irradiation. The enhancement of cell inhibition was dependent on the CD4+ T cell dose (see FIG. 27 A, see FIG. 27 B for transwell experiment) or soluble factors contained within the supernatant from activated CD4+ T cells (see FIG. 27 C).

This notion is confirmed by another cervical cancer cell line Caski (see FIG. 27 D), glioma cancer cell line U373 (see FIG. 27 E), lung cancer cell line A549 (see FIG. 27 F), breast cancer cell line MCF-7 (see FIG. 27 G), sarcoma cancer cell line 6647 (see FIG. 27 H) and prostate cancer cell line DU145 (see FIG. 27 I). Values are means±SEM. Similar results were found in at least two to three other independent experiments.

Thus, activated CD4+ T cells or their supernatants pre-sensitized tumor cells of various histologies to the apoptotic effects of radiation.

### Experiment 6

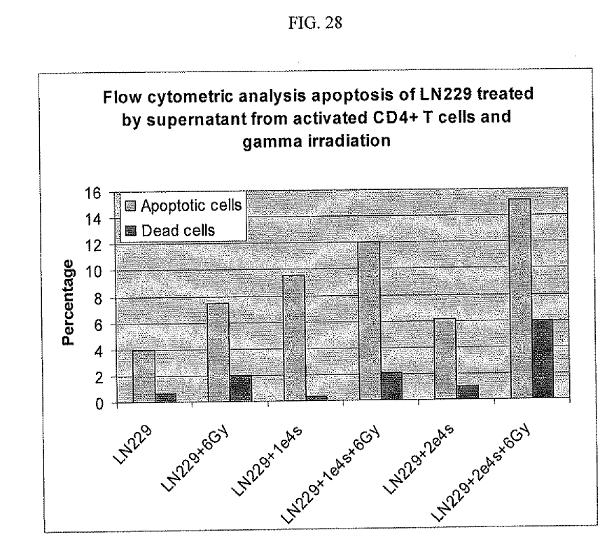

Experiments were conducted to determine which of the primary two mechanisms (apoptosis and necrosis) caused the cell inhibition for cells pre-sensitized with activated CD4+ T cells or their supernatant. The LN229 cells were plated at 4×10⁴ cells per well at 0.8 ml RPMI 1640 complete medium in a 24-well plate overnight. The following day, the tumor cells were treated by 0.8 ml RPMI 1640 complete medium containing different doses of activated CD4+ T cells via transwell (see FIG. 28). Forty-eight hours later, the tumor cells were collected and irradiated with gamma radiation at 4 Gy. The irradiated tumor cells were plated at 1×10⁵ cells per well in 1 ml of 0-25% supernatant derived from activated CD4+ T cells in a 24-well plate for 2 days. The tumor cells were stained with anti-annexin-V-FITC and PI, and were analyzed via flow cytometric analysis to determine the percentage of apoptotic cells and dead cells.

As shown in FIG. 28, the cell inhibition by activated CD4+ T cells and radiation is through apoptosis. The amount of apoptosis was dependent upon both the CD4+ T cell dose and the gamma radiation dose. Similar results were observed in another independent experiment.

### Experiment 7

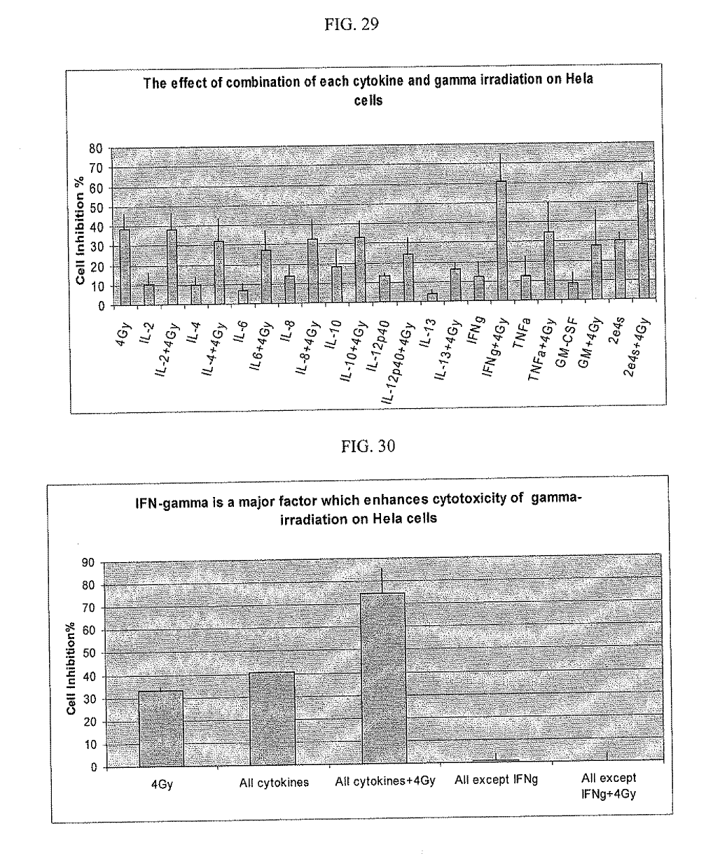

Hela cells were pre-sensitized for 2 days with each cytokine listed on FIG. 29. The pre-sensitized Hela cells were thereafter trypsinzed, washed, and irradiated with 4 Gy of gamma radiation. The irradiated cells were plated in 96-well plate at 5000 cells per well in 200 μl of complete medium. Cell viability was measured by WST-1 reagent on day 5.

The results show that IFN-γ is a major factor contributing to the enhanced effect of gamma irradiation (see FIG. 29). Similar results were found in another independent experiment.

### Experiment 8

Hela cells were pre-sensitized for 2 days by individual cytokines, combinations or all cytokines. The pre-sensitized Hela cells were thereafter harvested, washed, and irradiated with 4 Gy of gamma radiation. The irradiated cells were plated in 96-well plate at 5000 cells per well in 200 μl of RPMI 1640 medium. Cell viability was measured by WST-1 assay on day 5.

The result again showed that IFN-γ contributes to enhance cytotoxicity of gamma irradiation (see FIG. 30). Similar results were found in another independent experiment.

### Experiment 9

Hela cells were pre-sensitized with IFN-γ, and IFN-γ in combination with other cytokines for 2 days. The pre-sensitized Hela cells were then cultured with fresh complete RPMI 1640 for another 3 days. Cell viability was measured by WST-1 assay on day 5.

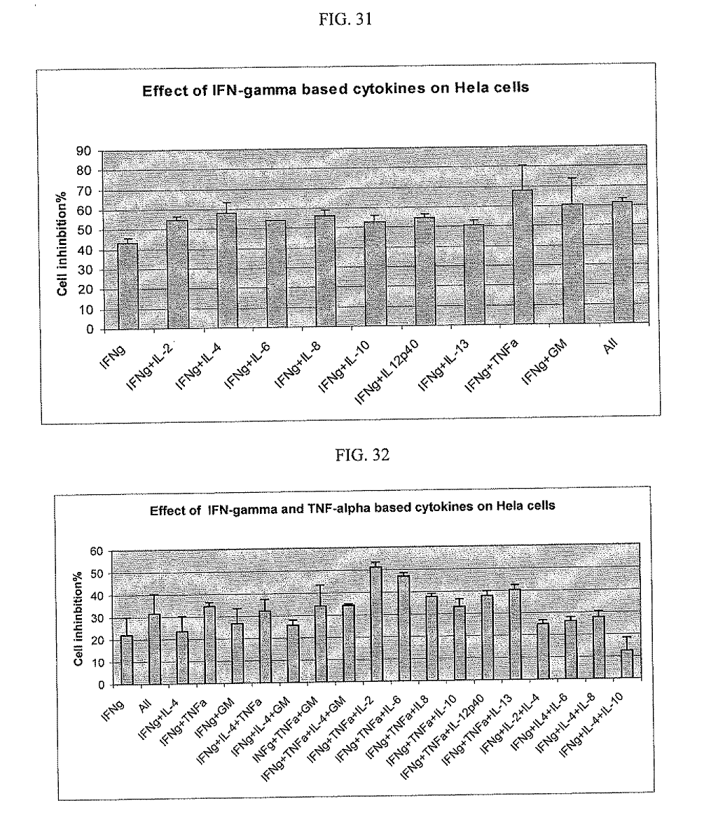

The results show that IFN-γ contributes to enhance cytotoxicity of gamma irradiation. Additionally, the results show that TNF-α and GM-CSF each have an added effect on the maximum cytotoxic effect of IFN-γ on Hela cells (see FIG. 31).

### Experiment 10

Hela cells were pre-sensitized for 2 days by the cytokines or combination of cytokines listed in FIG. 32. The pre-sensitized Hela cells were cultured with fresh complete RPMI 1640 for another 3 days. Cell viability was measured by WST-1 assay on day 5.

The results show that pre-sensitizing tumor cells with IFN-γ/TNF-α combined with Il-2 or Il-6 has the most noticeable increase in the maximum cytotoxic effect on Hela cells (see FIG. 32).

### Experiment 11

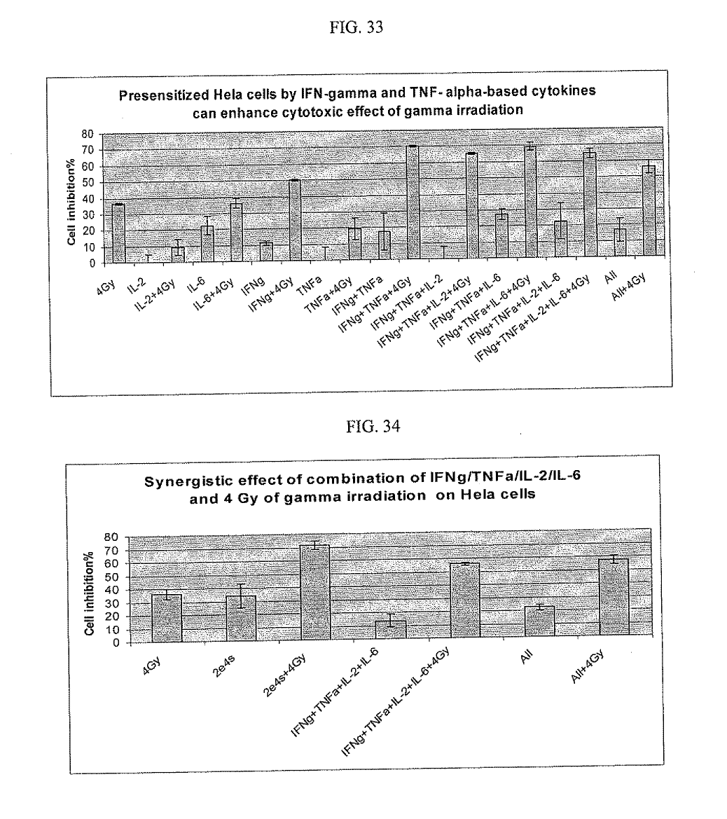

Hela cells were pre-sensitized with various combinations of cytokines identified in FIG. 33. The pre-sensitized Hela cells were made single suspension and irradiated by 4 Gy of gamma radiation. The irradiated cells were plated in 96-well plate at 5000 cells per well in 200 μl RPMI 1640 medium. Cell viability was measured by WST-1 assay on day 5.

Synergistic effect was found in samples that were pre-sensitized with [IFN-γ], [IFN-γ/TNF-α], [IFN-γ/TNF-α/IL-2], [IFN-γ/TNF-α/IL-6], [IFN-γ/TNF-α/IL-2/IL-6], and all cytokines (see FIG. 33). There were no significant differences between samples that were pre-sensitized with [IFN-γ/TNF-α], [IFN-γ/TNF-α/Il-2], [IFN-γ/TNF-α/Il-6], [IFN-γ/TNF-α/Il-2/Il-6], and the combination of all cytokines. Similar results were found in another independent experiment.

### Experiment 12

Hela cells were pre-sensitized for 2 days with CD4+ T cells, a combination of [IFN-γ/TNF-α/IL-2/Il-6], or a combination of all cytokines. The pre-sensitized cells were thereafter placed into a single suspension and irradiated by 4 Gy of gamma radiation. The irradiated cells were plated in 96-well plate at 5000 cells per well in 200 μl RPMI 1640 medium. Cell viability was measured by WST-1 assay on day 5.

As shown in FIG. 34 a synergistic effect was observed in samples pre-sensitized with supernatant derived from 2×10⁴ activated CD4+ T cells, the combination of [IFN-γ/TNF-α/IL-2/IL-6], and the combination of all cytokines. Compared with all cytokines combination, the combination of [IFN-γ/TNF-α/IL-2/IL-6] has the similar effect. Similar results were found in another independent experiment.

### Experiment 13

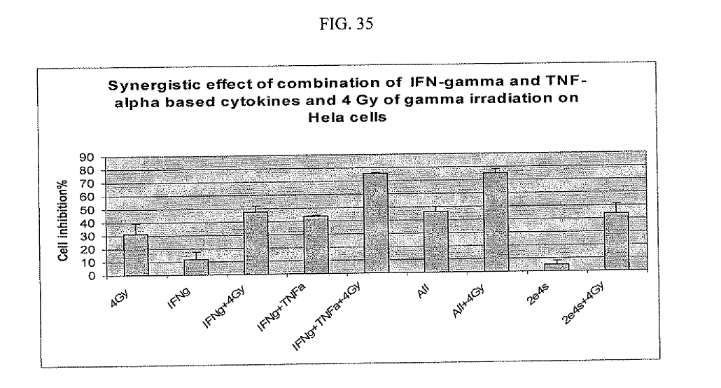

Another experiment was performed similar to experiment 16, except that [IFN-γ/TNF-α] was used instead of the combination of [IFN-γ/TNF-α/Il-2/Il-6]. The results were similar to the results observed in Experiment 13 (see FIG. 35).

### Experiment 14

An experiment was conducted to test the effect of radiotherapy on other cell lines that were pre-sensitized with cytokines. The other cell lines that were tested included lung cancer cell line A549, prostate cancer cell line DU145, glioma cell line U373, glioma cell line LN229 and cervical carcinoma cell line Caski+. These cell lines were pre-sensitized with the combination of [IFN-γ/TNF-α/Il-2/Il-6] and irradiated with gamma radiation similar to the procedure of Experiment 8-14.

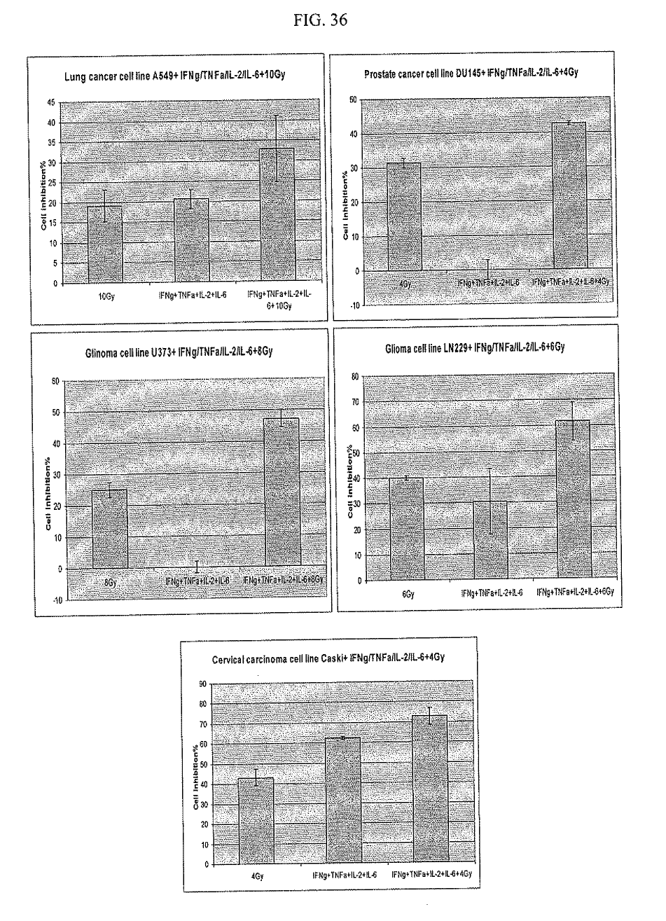

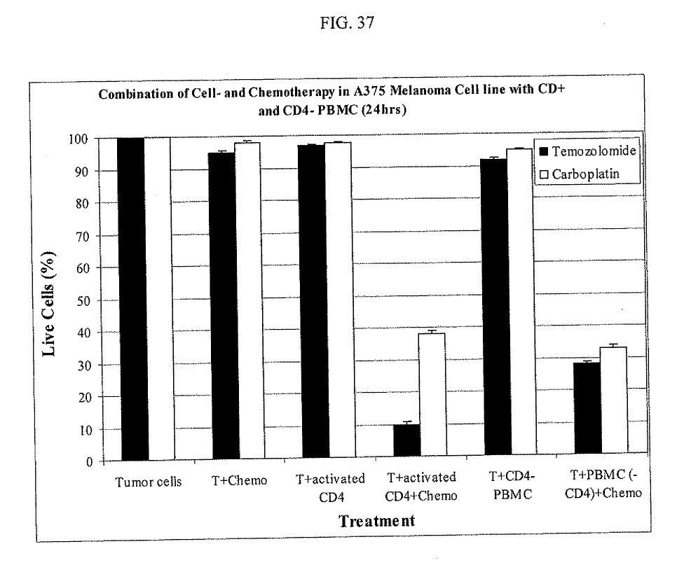

The results were similar to Experiments 8-14, thereby demonstrating the synergistic or positive effects of pre-sensitizing tumor cells in other cell lines (see FIG. 36).

### Experiment 15

An experiment was conducted to test the pre-sensitizing tumor cells with effect T+ CD4− peripheral blood mononuclear cells (“PBMC”). A375 melanoma cells with pre-sensitized with T+ CD4− PBMC or activated CD4+ T cells. A sample of the pre-sensitized cells was treated with either TMZ or carboplatin. The percentage of live cells were recorded for: untreated tumor cells, tumor cells treated with TMZ or carboplatin, tumor cells treated with CD4+ T cells or T+ CD4− PBMC, and tumor cells pre-sensitized with CD4+ T cells or T+ CD4− PBMC and treated with TMZ or carbo. The results (see FIG. 7) indicate that pre-sensitized tumor cells T+ CD4− PBMC also increase or induce apoptosis of tumor cells treated with radiotherapy.

### Experiment 16

Female athymic nude mice were injected subcutaneously with 5×10⁶ cell of A375 melanoma cells or MDA-MB 231 human breast cancer cells in the right flank that were suspended in PBS (100 to 200 μl). The tumors were allowed to develop to approximately 0.3 to 0.5 cm. The mice were then randomized into groups of 8 mice each. On day 0, the mice received either no treatment or ex-vivo activated human CD4+ T cells that were suspended in 50 μl of PBS intratumorally. Forty-eight hours later, TMZ (for mice injected with A375 melanoma cell line) or taxol (for mice injected with MDA-MB 231 human breast cancer line). The TMZ was dissolved in 100% DMSO (40 mg/ml), diluted in PBS (5 mg/ml) and administered intraperitoneally at a volume of 100 to 200 μl (a dose commonly used for in vivo preclinical studies—100 mg/kg) for 5 days. Taxol was diluted in PBS and used at 10 mg/kg for 5 days. The entire treatment cycle was repeated once.

Tumor growth was measured three times per week by a digital caliper. Survival was also recorded.

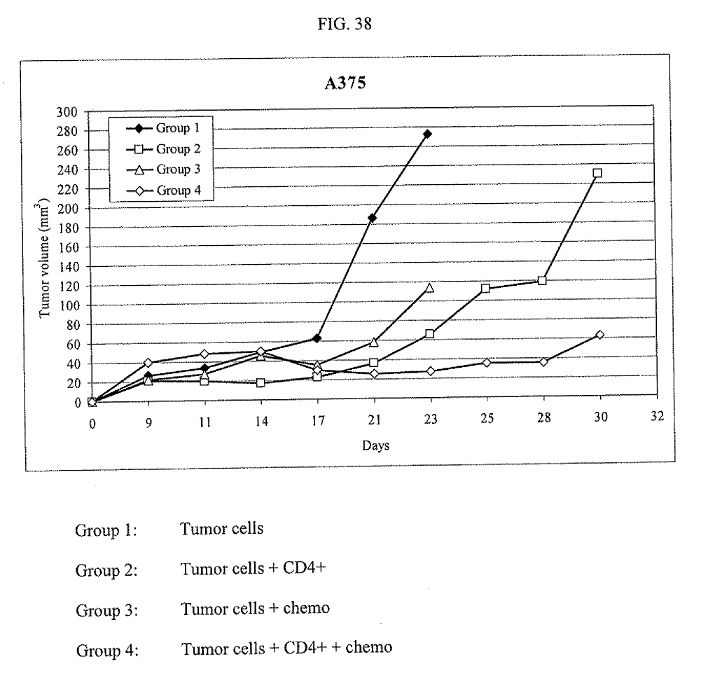

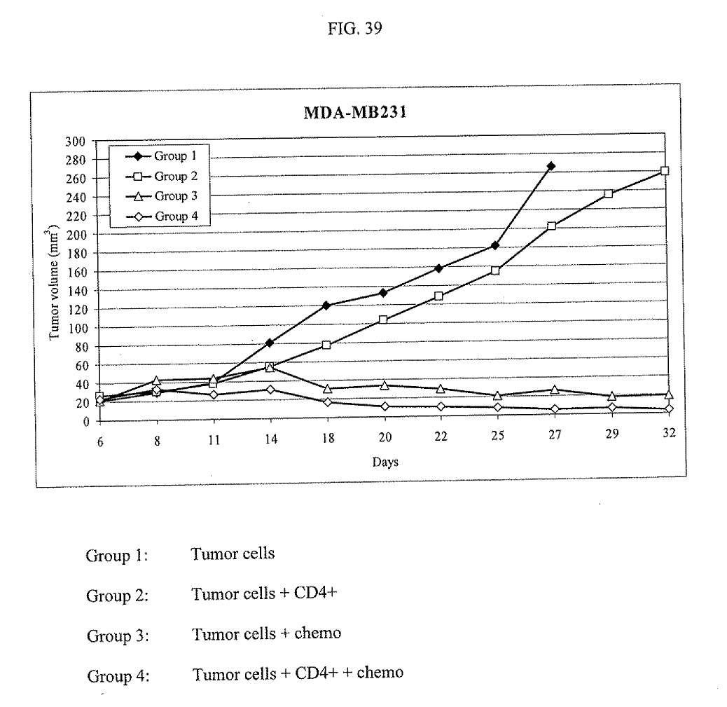

The tumors that were pre-sensitized with activated CD4+ T cells and treated with either TMZ or taxol were statistically and significantly smaller than the tumors that were not treated, treated with CD4+ T cells alone, or treated with TMZ or taxol alone (see FIG. 38 for A375 cell line, and see FIG. 39 for MDA-MB 231 cell line).

### V. CONCLUSION

Based on these results, it is confirmed that pre-sensitizing tumor cells or a subject having tumor cells with a pre-sensitizing agent can improve the effectiveness of chemotherapy or radiotherapy. The improvement may be at least about a two fold and up to about a ten fold.

Although the present invention has been described in considerable detail with reference to preferred embodiments thereof, however, other embodiments are possible for those skilled in the art and various modifications and variations can be made to the present invention without departing from the scope or spirit of the invention. In view of the foregoing, it is intended that the present invention cover modifications and variations of this invention provided they fall within the scope of the following claims and their equivalents. It is therefore intended that this invention be limited only as indicated by the appended claims.

The invention claimed is:

1. A method of treating cancer comprising the steps of:
administering at least one cytokine to a subject; and
administering a chemotherapeutic drug to the subject, thereby increasing or inducing apoptosis of a tumor cell.

2. The method according to claim 1, wherein the cytokine is selected from the group consisting of Il-2, Il-4, Il-6, Il-8, Il-10, Il-13, IFN-γ, TNF-α, Il-12p40, and GM-CSF.

3. The method according to claim 1, wherein the cytokine is selected from the group consisting of Il-10, Il-13, IFN-γ, TNF-αIl-12p40, and GM-CSF.

4. The method according to claim 1, wherein the cytokine comprises IFN-γ, TNF-α and GM-CSF.

5. The method according to claim 1, wherein the cytokine comprises IFN-γ.

6. The method according to claim 5, wherein the cytokine further comprises TNF-α.

7. The method according to claim 1, wherein the chemotherapeutic drug is selected from the group consisting of dacarbazine, temozolomide, carboplatin, paclitaxel, cisplatin, vinblastine, and fluorouracil.

8. A method of treating cancer comprising the steps of:
administering a pre-sensitizing agent to a subject having at least one tumor cell; and
administering a chemotherapeutic drug to the subject, thereby increasing or inducing apoptosis of the tumor cell.

9. The method according to claim 8, wherein the pre-sensitizing agent is a supernatant derived from at least one activated immune cell.

10. The method according to claim 8, wherein the pre-sensitizing agent is at least one activated immune cell selected from the group consisting of CD4+ T cells, CD8+ T cells, natural killer cells, natural killer T cells, B cells, dendritic cells, peripheral blood mononuclear cells, neutrophils, monocytes and macrophages.

11. The method according to claim 10, wherein the activated immune cell comprises an activated CD4+ T cell.

12. The method according to claim 8, wherein the chemotherapeutic drug is selected from the group consisting of dacarbazine, temozolomide, carboplatin, paclitaxel, cisplatin, vinblastine, and fluorouracil.

13. A method of treating cancer comprising the steps of:
administering a pre-sensitizing agent to a subject having at least one tumor cell;
allowing the pre-sensitizing agent to pre-sensitize the tumor cell; and
treating the subject or tumor cell with a radiotherapy.

14. The method according to claim 13, wherein the pre-sensitizing agent is at least one activated immune cell is selected from the group consisting of CD4+ T cells, CD8+ T cells, natural killer cells, natural killer T cells, B cells, dendritic cells, peripheral blood mononuclear cells, neutrophils, monocytes and macrophages.

15. The method according to claim 13, wherein the pre-sensitizing agent comprises an activated CD4+ T cell.

16. The method according to claim 13, wherein the pre-sensitizing agent is at least one cytokine.

17. The method according to claim 16, wherein the cytokine is selected from the group consisting of Il-2, Il-4, Il-6, Il-8, Il-10, Il-13, IFN-γ, TNF-α, Il-12p40, and GM-CSF.

18. The method according to claim 16, wherein the cytokine comprises IFN-γ.

19. The method according to claim 18, wherein the cytokine further comprises TNF-α.

20. The method according to claim 18, wherein the cytokine further comprises Il-2 or Il-6.

21. A method for enhancing the activity of a chemotherapeutic drug administered in combination with a pre-sensitizing agent and administering said chemotherapeutic drug to the subject containing said tumor cell, wherein said administering is effective to produce at least about a 2-fold and up to about a 10-fold reduction in vivo tumor volume relative to that provided by administering the chemotherapeutic drug alone.

22. The method according to claim 21, wherein the chemotherapeutic drug is selected from the group consisting of dacarbazine, temozolomide, carboplatin, paclitaxel, cisplatin, vinblastine, and fluorouracil.

23. A kit for enhancing the activity of a cancer treatment comprising a pre-sensitizing agent and a chemotherapeutic agent.

24. The kit according to claim 23, wherein the pre-sensitizing agent is administered to a subject prior to the administration of the chemotherapeutic agent.
